(19) United States
(12) Patent Application Publication
(10) Pub. No.: US 2014/0058132 A1
(43) Pub. Date: Feb. 27, 2014

(54) METHOD FOR PRODUCING ACETYLENEDICARBOXYLIC ACID FROM ACETYLENE AND CARBON DIOXIDE

(71) Applicant: BASF SE, Ludwigshafen (DE)

(72) Inventors: Matthias Arndt, Kaiserslautern (DE); Lukas J. Goossen, Kaiserslautern (DE)

(73) Assignee: BASF SE, Ludwigshafen (DE)

(21) Appl. No.: 13/968,701

(22) Filed: Aug. 16, 2013

Related U.S. Application Data
(60) Provisional application No. 61/692,728, filed on Aug. 24, 2012.

(51) Int. Cl. C07C 51/15

(52) U.S. Cl. CPC C07C 51/15
USPC 562/550

(57) ABSTRACT

The invention relates to a method for producing acetylenedicarboxylic acid by reaction of acetylene with carbon dioxide, wherein the reaction is carried out in the presence of a silver or copper salt and an amine base.

The invention relates to a method for producing acetylenedicarboxylic acid by reaction of acetylene with carbon dioxide.

According to an historic method, dating back to the year 1877, acetylenedicarboxylic acid is formed from meso-dibromosuccinic acid by alkaline elimination.

The formation of acetylenedicarboxylic acid with a yield of 34% by reacting dilithium carbide, obtained from reaction of vinyl bromide with butyllithium, with carbon dioxide in ether, where the carbon dioxide is added as dry ice, is described in E. C. Horning, J. Am Chem. Soc. 1945, 67, 1412-1422.

Acetylenedicarboxylic acid may be obtained in a yield of ca. 50% by the electrochemical oxidation of alkynols at the anode with lead oxide electrodes. An electrochemical method for producing acetylenedicarboxylic acid is described in V. Wolf, Chem. Ber. 1954, 87, 668-676. In this process, decarboxylation of the acetylenedicarboxylic acid, with formation of carbon dioxide and acetylene, occurs as a side reaction. The oxidation of alpha, omega-diols to dicarboxylic acids at nickel oxide anodes is described in J. Kaulen and H. J. Schäfer, Tetrahedron Lett. 1982, 38, 3299-3308. Electrochemical methods are however associated with a high energy expenditure and high costs.

The direct carboxylation of acetylene with 5 bar CO₂ in dimethylformamide in the presence of (4,7-diphenylphenanthroline)bis(triphenylphosphine)copper(l) and cesium carbonate is described in WO 2012/022801. This affords a mixture of acetylenemonocarboxylic acid and acetylenedicarboxylic acid, which are detected in the form of their n-hexyl esters in the reaction mixture. The conversion after a reaction time of two hours at 60° C. corresponds to a turnover number (TON) of ca. 1.8 for the formation of the acetylenedicarboxylic acid. WO 2012/022801 further discloses the carboxylation of terminal alkynes to the corresponding propiolic acids in the presence of phenanthroline-phosphine-copper(l) complexes and cesium carbonate. WO 2011/075087 discloses the carboxylation of terminal alkynes with CO₂ in the presence of a copper compound and an amine base.

The direct carboxylation of acetylene to acetylenedicarboxylic acids has proven to be difficult, since acetylene has only a low solubility in organic solvents and carbon dioxide, as co-reactant, due to its considerably higher solubility in organic solvents, additionally makes it difficult for acetylene to accumulate in the reaction medium. The salts of acetylenemonocarboxylic acid initially formed also have only a low solubility, which hinders the second carboxylation step to give the acetylenedicarboxylic acid, such that product mixtures of the acetylenemonocarboxylic acid and the acetylenedicarboxylic acid are formed. Furthermore, the acetylenecarboxylic acids, particularly acetylenedicarboxylic acid, tend to decarboxylate in solution, and the decarboxylation is moreover catalyzed by silver and copper complexes. Also, acetylene is prone to polymerization or vinylation reactions, which can additionally reduce the yield.

The carboxylation of acetylene in the presence of copper or silver compounds carries the additional risk of the formation of explosive copper or silver acetylides. Therefore, it is desirable to minimize the amount of catalyst used. This risk always exists when acetylene is handled under pressure. The less catalyst is used, the lower this risk is.

Also, the use of stoichiometric amounts of inorganic bases is disadvantageous in the method described in WO 2012/022801, due to the accumulation of salts, in a process carried out on an industrial scale. In order to be able to hydrogenate the acetylenedicarboxylic acid formed to the valuable target compound butane-1,4-diol in a subsequent step, the acetylenedicarboxylic acid must be separated from cesium carbonate and the other cesium salts present after the reaction.

The object of the invention is to make available a simple-to-perform method for producing acetylenedicarboxylic acid by direct carboxylation of acetylene. A particular object of the invention is to make available such a method that is characterized by a high turnover, based on the amount of catalyst used. It is a further object of the invention to make available such a method that obviates the need to use inorganic bases.

The object is achieved by a method for producing acetylenedicarboxylic acid by reaction of acetylene with carbon dioxide, wherein the reaction is carried out in the presence of a silver or copper salt and an amine base.

Surprisingly it has been found that the direct carboxylation of acetylene to acetylenedicarboxylic acid in the presence of a copper or silver salt also proceeds in the absence of an inorganic base such as cesium carbonate, if conducted in the presence of an amine base. This is even more surprising since amine bases coordinate very tightly to copper or silver and can block coordination sites for acetylene and CO₂. Since the solubility of carbon dioxide and particularly of acetylene is very low under the reaction conditions, it was to be expected that the copper or silver catalyst is deactivated by the large excess of an amine base in comparison to the small amount of dissolved acetylene. Furthermore, amines are carboxylated in the presence of CO₂ to form carbamates, which reduces their basicity. It is further surprising that the acetylenedicarboxylic acid formed under the reaction conditions (low partial pressure of carbon dioxide and high temperature) is stable and does not decarboxylate.

The direct carboxylation of acetylene to acetylenedicarboxylic acid takes place in the presence of silver or copper catalysts and an amine base. Suitable silver catalysts are chosen from the group consisting of elemental silver, colloidal silver particles, which may optionally comprise stabilizing additives such as phosphine ligands, dimethyl sulfoxide and/or polyvinylpyrrolidone, silver(l) halides such as AgF, AgCl, AgBr and Agl, silver nitrate, silver tetrafluoroborate, silver trifluoromethanesulfonate, silver carboxylate (silver acetate), silver hexafluorophosphate, silver oxide, silver sulfate, silver hexafluoroantimonate, silver p-toluenesulfonate and silver carbonate. Preference is given to silver(l) iodide Agl, silver(l) nitrate AgNO₃ and silver tetrafluoroborate AgBF₄, particular preference to silver(l) nitrate.

Suitable copper catalysts are elemental copper and colloidal copper particles and copper salts chosen from the group consisting of copper(l) halides such as CuF, CuCl, CuBr and Cul, copper(l) cyanide, copper tetrafluoroborate, copper trifluoromethanesulfonate, copper acetate, copper hexafluorophosphate, copper oxide, copper sulfate, copper hexafluoroantimonate, copper p-toluenesulfonate and copper carbonate. Preference is given to copper(l) iodide Cul and copper(l) cyanide CuCN, particular preference to Cul.

In one embodiment of the invention the reaction of acetylene with carbon dioxide is carried out in the presence of a silver salt, particularly AgNO₃. In a further embodiment of the invention the reaction of acetylene with carbon dioxide is carried out in the presence of a copper salt, particularly Cul.

Suitable amine bases are amine bases which are liquid at the reaction temperature, such as alkylamines, particularly tri-C₃-C₆-alkylamines such as tripropylamine and tributylamine, alkanolamines, particularly mono-, di- and tri-C₂-C₄-alkanolamines such as mono-, di- and triethanolamine, and particularly heterocyclic amine bases such as N-methylpiperidine, N-methylpiperidone, N-methylmorpholine, N-methyl-2-pyrrolidone, but above all diazabicyclononene (DBN) and diazabicycloundecene (DBU).

Particularly preferred amine bases are 1,5-diazabicyclo[4.3.0]non-5-ene (DBN) and 1,8-diazabicyclo[5.4.0]undec-7-ene (DBU).

The reaction may be carried out in the presence of a solvent. Suitable solvents are, for example, dimethyl sulfoxide (DMSO), dimethylformamide (DMF), water, NMP, dioxane, sulfolane and alcohols. Silver colloids can be pre-formed by heating in DMSO and then added to the reaction mixture. DMSO is a very good solvent; in addition, silver forms very fine colloids when heated therein.

The reaction may also be carried out in the absence of a separate solvent, in which case the silver salt or copper salt can be dissolved in the amine base.

The reaction is carried out generally at a total pressure (acetylene and carbon dioxide) of 1 to 50 bar, preferably 1 to 20 bar, and a temperature generally of 50 to 120° C., preferably 50 to 100° C. The molar ratio of carbon dioxide to acetylene is generally from 2:1 to 50:1, preferably from 5:1 to 20:1. With the method according to the invention a high turnover number (TON) is achieved.

In one embodiment of the invention the reaction is carried out at atmospheric pressure (1 bar). In a further embodiment the total pressure is maintained at 1 to 10 bar.

The acetylenedicarboxylic acid formed may subsequently be hydrogenated to butane-1,4-diol. The hydrogenation can be carried out without workup of the reaction mixture from acetylene carboxylation in the presence of the amine base.

The invention is further illustrated by the following examples.

### EXAMPLES

### Examples 1 to 4

The experiments were carried out in 100 mL headspace vials for gas chromatography, which were sealed with aluminum crimped caps and Teflon-coated butyl rubber septa.

In order to regulate the vial temperature, an 8 cm-high cylindrical aluminum block was used, whose diameter corresponded to that of the hotplate of a laboratory magnetic stirrer. The aluminum block was provided with 7 cm-deep wells with the diameter of the reaction vials and a well to accommodate a temperature probe.

A vacuum distributor was prepared for connection to a Schlenk line, to allow a simultaneous evacuating and filling of several vials. For this purpose, vacuum-tight Teflon tubes with a diameter of 3 mm were linked with adaptors to accept Luer-lock syringe needles at one end, and connected at the other end to a steel tube, which was linked via a vacuum tube to the Schlenk line.

The solid reactants were weighed into the reaction vials under air. 20 mm magnetic followers were added and the vials hermetically sealed with septum caps using a crimping tool. Subsequently the reaction vials were inserted into the wells of the aluminum block and linked to the vacuum distributor via hollow needles through the septum caps.

In order to establish an inert gas atmosphere in the reaction vials, they were evacuated three times consecutively and refilled twice with nitrogen and subsequently with carbon dioxide, Liquid reagents were added by syringe through the septum caps.

Examples 1 and 2 were carried out at pressure 5 bar. For this purpose, the needles of the vacuum distributor were withdrawn under a contraflow of CO₂ and the reaction vials transferred to an autoclave reactor. Subsequently, long, twisted cannulae were pierced through the septa of the reaction vials and the autoclave reactor was sealed. The atmosphere was exchanged via a gas vent by three vacuum-CO₂ cycles. Subsequently, the autoclave reactor was pressurized to 1 bar acetylene and 4 bar CO₂. It was stirred for 16 hours at 60° C. and ca. 700 revolutions per minute.

Examples 3 and 4 (reactant: 1-octyne) were carried out at pressure 20 bar. For this purpose, the needles of the vacuum distributor were removed under a contraflow of CO₂ and the reaction vials transferred to an autoclave reactor. Subsequently, long, twisted cannulae were pierced through the septa of the reaction vials and the autoclave reactor was sealed. The atmosphere was exchanged via a gas vent by three vacuum-CO₂ cycles. Subsequently, the autoclave reactor was pressurized to 20 bar CO₂. It was stirred for 16 hours at 40° C. and ca. 700 revolutions per minute.

After the end of the reaction time and cooling of the vials, either (1) a substance for esterification (1-bromohexane or methyl iodide) was added and n-tetradecane was injected as internal standard, or (2) the product was not reacted further. In this case mesitylene was added initially and NMP later as standard. Variant (1) was carried out in examples 3 and 4 and variant (2) in examples 1 and 2.

In the case of the esterification (1), the vials were opened after 30 minutes of stirring and, with the aid of disposable pipettes, 0.25 mL samples of the reaction mixture were transferred to 6 mL rolled-edge vials, which comprised 2.0 mL of ethyl acetate and 2.0 mL of an aqueous sodium hydrogen carbonate solution. The two phases were first mixed using the pipette and then the phases allowed to separate. Subsequently, 1.0 mL of each of the organic phases was filtered through 0.3 mL of anhydrous magnesium sulfate into a 2.0 mL glass sample vial and each was washed with 0.5 mL of solvent. For this, disposable pipettes which had been furnished with a cotton wool plug were used as filters.

In the case of the workup of the free acid (2), the DMSO solvent was first removed by filtration. The solid was taken up in 5.0 mL of distilled water with cooling in an ice bath and 50-100 μL of N-methylpyrrolidone (NMP) were added. The clear solution was transferred to a 2.0 mL glass sample vial for the determination of the turnover which was determined by HPLC relative to the internal standard and corrected with a response factor.

The results of the experiments are reproduced in the following table.

| | | | | | |
|---|---|---|---|---|---|
| | | | CO₂ | | |
| | | | Pressure | Yield | |
| Example | Catalyst | Base | [bar] | [%] | TON |

Experimemts with acetylene

| | | | | |
|---|---|---|---|---|
| 1 | 0.429 mg | 365 mg | 5 | 5 |
| | [0.0025 mmol] | [2.40 mmol] | | |
| | AgNO₃ | DBU | | |
| 2 | 1.07 mg | 365 mg | 5 | 7 |
| | [0.0055 mmol] | [2.40 mmol] | | |
| | ppm Cul | DBU | | |

Experiments with 1-octyne (comparison):

| | | | | | |
|---|---|---|---|---|---|
| 3 | 1.07 mg | 365 mg | 20 | 75 | 300 |
| | [0.0055 mmol] | [2.40 mmol] | | | |
| | Cul | DBU | | | |
| 4 | 0.0858 mg | 365 mg | 20 | 71 | 1418 |
| | [0.0005 mmol] | [2.40 mmol] | | | |
| | AgNO₃ | DBU | | | |

The results illustrate that the silver-catalyzed direct carboxylation of acetylene with CO₂ in the presence of amine bases is possible.

1. A method for producing acetylenedicarboxylic acid by reaction of acetylene with carbon dioxide, wherein the reaction is carried out in the presence of a silver or copper catalyst and an amine base.

2. The method according to claim 1 wherein the silver catalyst is selected from the group consisting of silver(l) halides, silver(l) nitrate and silver tetrafluoroborate.

3. The method according to claim 1 wherein the copper catalyst is chosen from the group consisting of copper(l) iodide and copper(l) cyanide.

4. The method according to claim 1 wherein the amine base is chosen from the group consisting of 1,5-diazabicyclo[4.3.0]non-5-ene (DBN) and 1,8-diazabicyclo[5.4.0]undec-7-ene (DBU).

5. The method according to claim 1 wherein the reaction is carried out in a solvent.

6. The method according to claim 1 wherein the reaction is carried out at a total pressure of 1-50 bar and a temperature of 50 to 120° C.

7. The method according to claim 1 wherein the reaction is carried out with a molar ratio of carbon dioxide to acetylene of 2:1 to 50:1.
